(19) United States
(12) Patent Application Publication
(10) Pub. No.: US 2009/0023756 A1
(43) Pub. Date: Jan. 22, 2009

(54) SUBSTITUTED QUINAZOLINES AS PDE10 INHIBITORS

(75) Inventors: Martin Patrick Allen, Voluntown, CT (US); Thomas Allen Chappie, Old Lyme, CT (US); John Michael Humphrey, Mystic, CT (US); Spiros Liras, Stonington, CT (US)

Correspondence Address: PFIZER INC;Steve T. Zelson, 150 EAST 42ND STREET, 5TH FLOOR - STOP 49, NEW YORK, NY, 10017-5612, US

(73) Assignee: Pfizer Inc

(21) Appl. No.: 12/279,869

(22) PCT Filed: Feb. 9, 2007
(86) PCT No.: PCT/IB07/00411

§ 371 Date: Aug. 19, 2008

Related U.S. Application Data
(60) Provisional application No. 60/776,112, filed on Feb. 23, 2006.

(51) Int. Cl. A61K 31/517; A61P 25/00; C07D 471/04; A61K 31/519; C07D 471/14

(52) U.S. Cl. 514/264.1; 544/284; 544/279; 514/266.21

(57) ABSTRACT

The invention pertains to substituted quinazoline compounds of structures (I) and (II) that serve as effective phosphodiesterase (PDE) inhibitors. The invention also relates to compounds which are selective inhibitors of PDE-10. The invention further relates to intermediates for preparation of such compounds; pharmaceutical compositions comprising such compounds; and the use of such compounds in methods for treating certain central nervous system (CNS) or other disorders. The invention relates also to methods for treating neurodegenerative and psychiatric disorders, for example psychosis and disorders comprising deficient cognition as a symptom. (I, II).

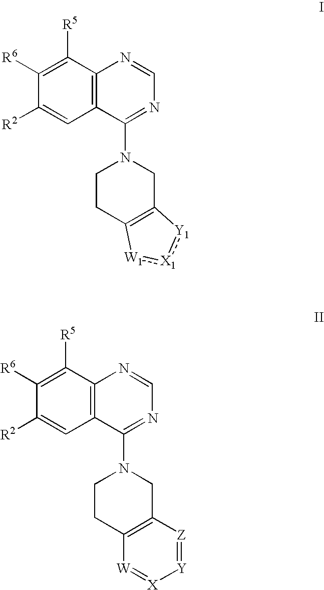

### FIELD OF THE INVENTION

The invention pertains to substituted quinazoline compounds that serve as effective phosphodiesterase (PDE) inhibitors. The invention also relates to compounds that are selective inhibitors of PDE-10. The invention further relates to intermediates for preparation of such compounds; pharmaceutical compositions comprising such compounds; and the use of such compounds in methods for treating certain central nervous system (CNS) or other disorders. The invention relates also to methods for treating neurodegenerative and psychiatric disorders, for example psychosis and disorders comprising deficient cognition as a symptom.

### BACKGROUND OF INVENTION

Phosphodiesterases (PDEs) are a class of intracellular enzymes involved in the hydrolysis of the nucleotides cyclic adenosine monophosphate (cAMP) and cyclic guanosine monophosphates (cGMP) into their respective nucleotide monophosphates. The cyclic nucleotides cAMP and cGMP are synthesized by adenylyl and guanylyl cyclases, respectively, and serve as secondary messengers in several cellular pathways.

The cAMP and cGMP function as intracellular second messengers regulating a vast array of intracellular processes particularly in neurons of the central nervous system. In neurons, this includes the activation of cAMP and cGMP-dependent kinases and subsequent phosphorylation of proteins involved in acute regulation of synaptic transmission as well as in neuronal differentiation and survival. The complexity of cyclic nucleotide signaling is indicated by the molecular diversity of the enzymes involved in the synthesis and degradation of cAMP and cGMP. There are at least ten families of adenylyl cyclases, two of guanylyl cyclases, and eleven of phosphodiesterases. Furthermore, different types of neurons are known to express multiple isozymes of each of these classes, and there is good evidence for compartmentalization and specificity of function for different isozymes within a given neuron.

A principal mechanism for regulating cyclic nucleotide signaling is by phosphodiesterase-catalyzed cyclic nucleotide catabolism. There are 11 known families of PDEs encoded by 21 different genes. Each gene typically yields multiple splice variants that further contribute to the isozyme diversity. The PDE families are distinguished functionally based on cyclic nucleotide substrate specificity, mechanism(s) of regulation, and sensitivity to inhibitors. Furthermore, PDEs are differentially expressed throughout the organism, including in the central nervous system. As a result of these distinct enzymatic activities and localization, different PDEs' isozymes can serve distinct physiological functions. Furthermore, compounds that can selectively inhibit distinct PDE families or isozymes may offer particular therapeutic effects, fewer side effects, or both.

PDE10 is identified as a unique family based on primary amino acid sequence and distinct enzymatic activity. Homology screening of EST databases revealed mouse PDE10A as the first member of the PDE10 family of PDEs (Fujishige et al., J. Biol. Chem. 274:18438-18445, 1999; Loughney, K. et al., Gene 234:109-117, 1999). The murine homologue has also been cloned (Soderling, S. et al., Proc. Natl. Acad. Sci. USA 96:7071-7076, 1999) and N-terminal splice variants of both the rat and human genes have been identified (Kotera, J. et al., Biochem. Biophys. Res. Comm. 261:551-557, 1999; Fujishige, K. et al., Eur. J. Biochem. 266:1118-1127, 1999). There is a high degree of homology across species. The mouse PDE10A1 is a 779 amino acid protein that hydrolyzes both cAMP and cGMP to AMP and GMP, respectively. The affinity of PDE10 for cAMP (Km=0.05 μM) is higher than for cGMP (Km=3 μM). However, the approximately 5-fold greater Vmax for cGMP over cAMP has lead to the suggestion that PDE10 is a unique cAMP-inhibited cGMPase (Fujishige et al., J. Biol. Chem. 274:18438-18445, 1999).

The PDE 10 family of polypeptides shows a lower degree of sequence homology as compared to previously identified PDE families and has been shown to be insensitive to certain inhibitors that are known to be specific for other PDE families. U.S. Pat. No. 6,350,603, incorporated herein by reference.

PDE10 also is uniquely localized in mammals relative to other PDE families. mRNA for PDE10 is highly expressed only in testis and brain (Fujishige, K. et al., Eur J. Biochem. 266:1118-1127, 1999; Soderling, S. et al., Proc. Natl. Acad. Sci. 96:7071-7076, 1999; Loughney, K. et al., Gene 234:109-117, 1999). These initial studies indicated that within the brain PDE10 expression is highest in the striatum (caudate and putamen), n. accumbens, and olfactory tubercle. More recently, a detailed analysis has been made of the expression pattern in rodent brain of PDE10 mRNA (Seeger, T. F. et al., Abst. Soc. Neurosci. 26:345.10, 2000) and PDE10 protein (Menniti, F. S., Stick, C. A., Seeger, T. F., and Ryan, A. M., Immunohistochemical localization of PDE10 in the rat brain. William Harvey Research Conference ‘Phosphodiesterase in Health and Disease’, Porto, Portugal, Dec. 5-7, 2001).

A variety of therapeutic uses for PDE inhibitors have been reported including obtrusive lung disease, allergies, hypertension, angina, congestive heart failure, depression and erectile dysfunction (WO 01/41807 A2, incorporated herein by reference).

U.S. Pat. No. 6,538,029 B1 describes the use of PDE10 inhibitors for the treatment of renal cell carcinoma.

The use of selected benzimidazole and related heterocyclic compounds in the treatment of ischemic heart conditions has been disclosed based upon inhibition of PDE associated cGMP activity. U.S. Pat. No. 5,693,652, incorporated herein by reference.

United States Patent Application Publication No. 2003/0032579 discloses a method for treating certain neurologic and psychiatric disorders with the selective PDE10 inhibitor papaverine. In particular, the method relates to psychotic disorders such as schizophrenia, delusional disorders and drug-induced psychosis; to anxiety disorders such as panic and obsessive-compulsive disorder; and to movement disorders including Parkinson's disease and Huntington's disease.

### SUMMARY OF THE INVENTION

The present invention provides for a compound of formulas I or II,

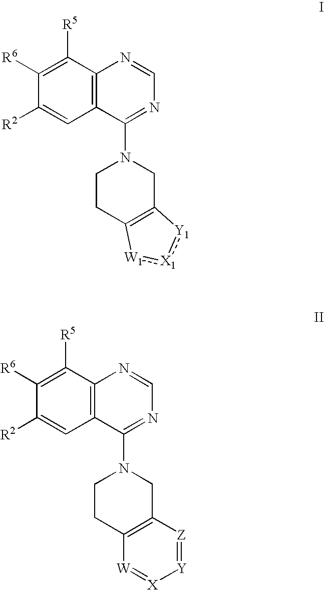

or a pharmaceutically acceptable salt,

wherein the rings containing W₁, X₁ and Y₁, and W, X, Y and Z, or tautomers thereof, are aromatic or heteroaromatic;

wherein X₁ is N or CR;

W₁ and Y₁ are each independently N, NR¹ or CR;

wherein the solid and dashed lines between W₁, X₁ and Y₁ represent single or double bonds, provided that one is a single bond and the other is a double bond;

wherein W, X, Y, Z are each independently N or CR;

wherein R², R⁵ and R⁶ are each independently hydrogen, halogen, —CN, —COOH, —COOR³, —CONR³R⁴, —COR³, —NR³R⁴, —OH, —NO₂, —(C₆-C₁₄)aryl, 5 to 12 membered heteroaryl, (C₁-C₈)alkyl, (C₁-C₈)alkoxy (C₂-C₈)alkenyl, (C₂-C₈)alkenyloxy (C₂-C₈)alkynyl or (C₃-C₈) cycloalkyl; wherein said alkyl, alkoxy, alkenyl, alkenyloxy, alkynyl and cycloalkyl are optionally independently substituted with from 1 to 5 halogens; and when R², R⁵ and R⁶ are independently alkoxy, alkenyloxy or alkyl, R² and R⁶ or R⁵ and R⁶ may optionally be connected to form a 5 to 8 membered ring;

wherein each R is independently H, —COOR³, —CONR³R⁴, —COR⁴, —NR³R⁴, —NHCOR³, —OH, —HNCOOR³, —CN, —HNCONHR⁴, (C₁-C₈)alkyl, (C₃-C₈)cycloalkyl, (C₁-C₈)alkoxy, phenyl, naphthyl, or a 5- to 8-membered heteroaryl ring, wherein said phenyl, naphthyl, or a 5- to 8-membered heteroaryl ring may be optional fused to the ring in which R is attached and wherein said alkyl, cycloalkyl, alkoxy, phenyl, naphthyl, heteroaryl or fused ring may optionally be substituted with from one to three substituents independently selected from (C₁-C₈)alkyl, (C₁-C₈)alkoxy, halogen, halo(C₁-C₈)alkyl, halo(C₁-C₈)alkoxy, (C₁-C₈)hydroxyalkyl, (C₁-C₈)alkoxy-(C₁-C₈)alkyl, (C₃-C₈)hydroxycycloalkyl, (C₃-C₈)cycloalkoxy, (C₁-C₈)alkoxy-(C₃-C₈)cycloalkyl, heterocycloalkyl, hydroxyheterocycloalkyl, and (C₁-C₈)alkoxy-heterocycloalkyl, wherein each cycloalkyl or heterocycloalkyl moiety may be independently substituted with from one to three (C₁-C₆)alkyl or benzyl groups;

wherein R¹ is H, —COOR³, —CONR³R⁴, —COR⁴, (C₁-C₈)alkyl, (C₃-C₈)cycloalkyl, phenyl, naphthyl, or a 5- to 8-membered heteroaryl ring, wherein said alkyl, cycloalkyl, phenyl, naphthyl, heteroaryl may optionally be substituted with from one to three substituents independently selected from (C₁-C₈)alkyl, (C₁-C₈)alkoxy, halogen, halo(C₁-C₈)alkyl, halo(C₁-C₈)alkoxy, (C₁-C₈)hydroxyalkyl, (C₁-C₈)alkoxy-(C₁-C₈)alkyl, (C₃-C₈)hydroxycycloalkyl, (C₃-C₈)cycloalkoxy, (C₁-C₈)alkoxy-(C₃-C₈)cycloalkyl, heterocycloalkyl, hydroxyheterocycloalkyl, and (C₁-C₈)alkoxy-heterocycloalkyl, wherein each cycloalkyl or heterocycloalkyl moiety may be independently substituted with from one to three (C₁-C₆)alkyl or benzyl groups;

wherein R³ and R⁴ are each independently H, (C₁-C₈)alkyl, alkenyl, aryl or benzyl; or R³ and R⁴ together with the nitrogen to which they are attached form a 5- to 8-membered heteroalkyl ring.

In one aspect of the present invention, R is phenyl or pyridine fused to the ring in which R is attached; W₁ or Y₁ is N or NR¹ in Formula I; or W or Y is N in Formula II.

In another aspect of the present invention, R is further substituted by (C₁-C₄)alkoxy, (C₁-C₄)alkyl, (C₁-C₅)trifluoroalkyl or (C₁-C₅)trifluoroalkoxy.

In another aspect of the present invention, R¹ is H, (C₁-C₆)alkoxy, (C₁-C₆)alkyl, (C₁-C₅)trifluoroalkyl, (C₁-C₅)trifluoroalkoxy or phenyl; R¹ is H, (C₁-C₆)alkyl, (C₁-C₅)trifluoroalkyl, or phenyl and at least one of W, X, Y, Z are nitrogen in Formula II.

In another aspect of the present invention R⁶ and R² are each independently (C₁-C₆)alkoxy.

In another aspect of the present invention R⁶ and R² are each-independently ethoxy or methoxy.

In another aspect of the present invention the compound is selected from formula I and R⁶ and R² are each independently (C₁-C₆)alkoxy.

In another aspect of the present invention the compound is selected from formula II and R⁶ and R² are each independently (C₁-C₆)alkoxy.

Compounds of Formulas I or II may have optical centers and therefore may occur in different enantiomeric and diastereomeric configurations. The present invention includes all enantiomers, diastereomers, and other stereoisomers of such compounds of Formulas I or II, as well as racemic compounds and racemic mixtures and other mixtures of stereoisomers thereof.

Pharmaceutically acceptable salts of the compounds of Formulas I or II include the acid addition and base salts thereof.

Suitable acid addition salts are formed from acids which form non-toxic salts. Examples include, but are not limited to, the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mandelates mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, salicylate, saccharate, stearate, succinate, sulfonate, stannate, tartrate, tosylate, trifluoroacetate and xinofoate salts.

Suitable base salts are formed from bases which form non-toxic salts. Examples include, but are not limited to, the aluminium, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulphate and hemicalcium salts.

For a review on suitable salts, see Handbook of Pharmaceutical Salts: Properties, Selection, and Use by Stahl and Wermuth (Wiley-VCH, 2002).

Pharmaceutically acceptable salts of compounds of Formulas I or II may be prepared by one or more of three methods:

(i) by reacting the compound of Formulas I or II with the desired acid or base;

(ii) by removing an acid- or base-labile protecting group from a suitable precursor of the compound of Formulas I or II or by ring-opening a suitable cyclic precursor, for example, a lactone or lactam, using the desired acid or base; or

(iii) by converting one salt of the compound of Formulas I or II to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionization in the resulting salt may vary from completely ionised to almost non-ionised.

The compounds of the invention may exist in a continuum of solid states ranging from fully amorphous to fully crystalline. The term amorphous' refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterized by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (‘melting point’).

The compounds of the invention may also exist in unsolvated and solvated forms. The term ‘solvate’ is used herein to describe a molecular complex comprising the compound of the invention and one or more pharmaceutically acceptable solvent molecules, for example, ethanol. The term ‘hydrate’ is employed when said solvent is water.

A currently accepted classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

The compounds of the invention may also exist in a mesomorphic state (mesophase or liquid crystal) when subjected to suitable conditions. The mesomorphic state is intermediate between the true crystalline state and the true liquid state (either melt or solution). Mesomorphism arising as the result of a change in temperature is described as ‘thermotropic’ and that resulting from the addition of a second component, such as Water or another solvent, is described as ‘Iyotropic’. Compounds that have the potential to form lyotropic mesophases are described as ‘amphiphilic’ and consist of molecules which possess an ionic (such as —COO⁻Na⁺, —COO⁻K⁺, or —SO₃⁻Na⁺) or non-ionic (such as —N⁻N⁺(CH₃)₃) polar head group. For more information, see Crystals and the Polarizing Microscope by N. H. Hartshorne and A. Stuart, 4th Edition (Edward Arnold, 1970).

Hereinafter all references to compounds of Formulas I or II include references to salts, solvates, multi-component complexes and liquid crystals thereof and to solvates, multi-component complexes and liquid crystals of salts thereof.

The compounds of the invention include compounds of Formulas I or II as hereinbefore defined, including all polymorphs and crystal habits thereof, prodrugs and isomers thereof (including optical, geometric and tautomeric isomers) as hereinafter defined and isotopically-labeled compounds of Formulas I or II.

As indicated, so-called ‘prodrugs’ of the compounds of Formulas I or II are also within the scope of the invention. Thus certain derivatives of compounds of Formulas I or II which may have little or no pharmacological activity themselves can, when administered into or onto the body, be converted into compounds of Formulas I or II having the desired activity, for example, by hydrolytic cleavage. Such derivatives are referred to as ‘prodrugs’. Further information on the use of prodrugs may be found in Pro-drugs as Novel Delivery Systems, Vol. 14, ACS Symposium Series (T. Higuchi and W. Stella) and Bioreversible Carriers in Drug Design, Pergamon Press, 1987 (Ed. E. B. Roche, American Pharmaceutical Association).

Prodrugs in accordance with the invention can, for example, be produced by replacing appropriate functionalities present in the compounds of Formulas I or II with certain moieties known to those skilled in the art as ‘pro-moieties’ as described, for example, in Design of Prodrugs by H. Bundgaard (Elsevier, 1985).

Some examples of prodrugs in accordance with the invention include, but are not limited to,

(i) where the compound of Formulas I or II contains a carboxylic acid functionality (—COOH), an ester thereof, for example, a compound wherein the hydrogen of the carboxylic acid functionality of the compound of Formula (I) is replaced by (C₁-C₈)alkyl;

(ii) where the compound of Formulas I or II contains an alcohol functionality (—OH), an ether thereof, for example, a compound wherein the hydrogen of the alcohol functionality of the compound of Formulas I or II is replaced by (C₁-C₆)alkanoyloxymethyl; and

(iii) where the compound of Formulas I or II contains a primary or secondary amino functionality (—NH₂ or —NHR where R≠H), an amide thereof, for example, a compound wherein, as the case may be, one or both hydrogens of the amino functionality of the compound of Formulas I or II is/are replaced by (C₁-C₁₀)alkanoyl.

Further examples of replacement groups in accordance with the foregoing examples and examples of other prodrug types may be found in the aforementioned references.

Moreover, certain compounds of Formulas I or II may themselves act as prodrugs of other compounds of Formulas I or II.

Also included within the scope of the invention are metabolites of compounds of Formulas I or II, that is, compounds formed in vivo upon administration of the drug. Some examples of metabolites in accordance with the invention include, but are not limited to,

(i) where the compound of Formulas I or II contains a methyl group, an hydroxymethyl derivative thereof (—CH₃->—CH₂OH):

(ii) where the compound of Formulas I or II contains an alkoxy group, an hydroxy derivative thereof (—OR—>—OH);

(iii) where the compound of Formulas I or II contains a tertiary amino group, a secondary amino derivative thereof (—NR¹R²—>—NHR¹ or —NHR²);

(iv) where the compound of Formulas I or II contains a secondary amino group, a primary derivative thereof (—NHR¹—>—NH₂);

(v) where the compound of Formulas I or II contains a phenyl moiety, a phenol derivative thereof (-Ph->-PhOH); and

(vi) where the compound of Formulas I or II contains an amide group, a carboxylic acid derivative thereof (—CONH₂—>COOH).

Compounds of Formulas I or II containing one or more asymmetric carbon atoms can exist as two or more stereoisomers. Where a compound of Formulas I or II contains an alkenyl or alkenylene group, geometric cis/trans (or Z/E) isomers are possible. Where structural isomers are interconvertible via a low energy barrier, tautomeric isomerism (‘tautomerism’) can occur. This can take the form of proton tautomerism in compounds of Formulas I or II containing, for example, an imino, keto, or oxime group, or so-called valence tautomerism in compounds that contain an aromatic moiety. It follows that a single compound may exhibit more than one type of isomerism.

Included within the scope of the present invention are all stereoisomers, geometric isomers and tautomeric forms of the compounds of Formulas I or II, including compounds exhibiting more than one type of isomerism, and mixtures of one or more thereof. Also included are acid addition or base salts wherein the counterion is optically active, for example, d-lactate or l-lysine, or racemic, for example, dl-tartrate or dl-arginine.

Cis/trans isomers may be separated by conventional techniques well known to those skilled in the art, for example, chromatography and fractional crystallisation.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of Formulas I or II contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and from 0 to 5% by volume of an alkylamine, typically 0.1% diethylamine. Concentration of the eluate affords the enriched mixture.

When any racemate crystallizes, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

While both of the crystal forms present in a racemic mixture have identical physical properties, they may have different physical properties compared to the true racemate. Racemic mixtures may be separated by conventional techniques known to those skilled in the art—see, for example, Stereochemistry of Organic Compounds by E. L. Eliel and S. H. Wilen (Wiley, 1994).

The present invention includes all pharmaceutically acceptable isotopically-labelled compounds of Formulas I or II wherein one or more atoms are replaced by atoms having the same atomic number, but an atomic mass or mass number different from the atomic mass or mass number which predominates in nature.

Examples of isotopes suitable for inclusion in the compounds of the invention include, but are not limited to, isotopes of hydrogen, such as ²H and ³H, carbon, such as ¹¹C, ¹³C and ⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of Formulas I or II, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds of Formulas I or II can generally be prepared by conventional techniques known to those skilled in the art or by processes analogous to those described in the accompanying Examples and Preparations using an appropriate isotopically-labeled reagent in place of the non-labeled reagent previously employed.

Pharmaceutically acceptable solvates in accordance with the invention include those wherein the solvent of crystallization may be isotopically substituted, e.g. D₂O, d₆-acetone, d₆-DMSO.

Specific embodiments of the present invention include the compounds exemplified in the Examples below and their pharmaceutically acceptable salts, complexes, solvates, polymorphs, steroisomers, metabolites, prodrugs, and other derivatives thereof, This invention also pertains to a pharmaceutical composition for treatment of certain psychotic disorders and conditions such as schizophrenia, delusional disorders and drug induced psychosis; to anxiety disorders such as panic and obsessive-compulsive disorder; and to movement disorders including Parkinson's disease and Huntington's disease, comprising an amount of a compound of Formulas I or II effective in inhibiting PDE 10.

In another embodiment, this invention relates to a pharmaceutical composition for treating psychotic disorders and condition such as schizophrenia, delusional disorders and drug induced psychosis; anxiety disorders such as panic and obsessive-compulsive disorder; and movement disorders including Parkinson's disease and Huntington's disease, comprising an amount of a compound of Formulas I or II effective in treating said disorder or condition.

Examples of psychotic disorders that can be treated according to the present invention include, but are not limited to, schizophrenia, for example of the paranoid, disorganized, catatonic, undifferentiated, or residual type; schizophreniform disorder; schizoaffective disorder, for example of the delusional type or the depressive type; delusional disorder; substance-induced psychotic disorder, for example psychosis induced by alcohol, amphetamine, cannabis, cocaine, hallucinogens, inhalants, opioids, or phencyclidine; personality disorder of the paranoid type; and personality disorder of the schizoid type.

Examples of movement disorders that can be treated according to the present invention include but are not limited to selected from Huntington's disease and dyskinesia associated with dopamine agonist therapy, Parkinson's disease, restless leg syndrome, and essential tremor.

Other disorders that can be treated according to the present invention are obsessive/compulsive disorders, Tourette's syndrome and other the disorders.

In another embodiment, this invention relates to a method for treating an anxiety disorder or condition in a mammal which method comprises administering to said mammal an amount of a compound of Formulas I or II effective in inhibiting PDE 10.

This invention also provides a method for treating an anxiety disorder or condition in a mammal which method comprises administering to said mammal an amount of a compound of Formulas I or II effective in treating said disorder or condition.

Examples of anxiety disorders that can be treated according to the present invention include, but are not limited to, panic disorder; agoraphobia; a specific phobia; social phobia; obsessive-compulsive disorder; post-traumatic stress disorder; acute stress disorder; and generalized anxiety disorder.

This invention further provides a method of treating a drug addiction, for example an alcohol, amphetamine, cocaine, or opiate addiction, in a mammal, including a human, which method comprises administering to said mammal an amount of a compound of Formulas I or II effective in treating drug addiction.

This invention also provides a method of treating a drug addiction, for example an alcohol, amphetamine, cocaine, or opiate addiction, in a mammal, including a human, which method comprises administering to said mammal an amount of a compound of Formulas I or II effective in inhibiting PDE10.

A “drug addiction”, as used herein, means an abnormal desire for a drug and is generally characterized by motivational disturbances such a compulsion to take the desired drug and episodes of intense drug craving.

This invention further provides a method of treating a disorder comprising as a symptom a deficiency in attention and/or cognition in a mammal, including a human, which method comprises administering to said mammal an amount of a compound of Formulas I or II effective in treating said disorder.

This invention also provides a method of treating a disorder or condition comprising as a symptom a deficiency in attention and/or cognition in a mammal, including a human, which method comprises administering to said mammal an amount of a compound of Formulas I or II effective in inhibiting PDE10.

This invention also provides a method of treating a disorder or condition comprising as a symptom a deficiency in attention and/or cognition in a mammal, including a human, which method comprises administering to said mammal an amount of a compound of Formulas I or II effective in treating said disorder or condition.

The phrase “deficiency in attention and/or cognition” as used herein in “disorder comprising as a symptom a deficiency in attention and/or cognition” refers to a subnormal functioning in one or more cognitive aspects such as memory, intellect, or learning and logic ability, in a particular individual relative to other individuals within the same general age population. “Deficiency in attention and/or cognition” also refers to a reduction in any particular individual's functioning in one or more cognitive aspects, for example as occurs in age-related cognitive decline.

Examples of disorders that comprise as a symptom a deficiency in attention and/or cognition that can be treated according to the present invention are dementia, for example Alzheimer's disease, multi-infarct dementia, alcoholic dementia or other drug-related dementia, dementia associated with intracranial tumors or cerebral trauma, dementia associated with Huntington's disease or Parkinson's disease, or AIDS-related dementia; delirium; amnestic disorder; post-traumatic stress disorder; mental retardation; a learning disorder, for example reading disorder, mathematics disorder, or a disorder of written expression; attention-deficit/hyperactivity disorder; and age-related cognitive decline.

This invention also provides a method of treating a mood disorder or mood episode in a mammal, including a human, comprising administering to said mammal an amount of a compound of Formulas I or II effective in treating said disorder or episode.

This invention also provides a method of treating obesity in a mammal, including a human, comprising administering to said mammal an amount of a compound of Formulas I or II effective in treating obesity.

This invention also provides a method of treating a mood disorder or mood episode in a mammal, including a human, comprising administering to said mammal an amount of a compound of Formulas I or II effective in inhibiting PDE10.

Examples of mood disorders and mood episodes that can be treated according to the present invention include, but are not limited to, major depressive episode of the mild, moderate or severe type, a manic or mixed mood episode, a hypomanic mood episode; a depressive episode with atypical features; a depressive episode with melancholic features; a depressive episode with catatonic features; a mood episode with postpartum onset; post-stroke depression; major depressive disorder; dysthymic disorder; minor depressive disorder; premenstrual dysphoric disorder; post-psychotic depressive disorder of schizophrenia; a major depressive disorder superimposed on a psychotic disorder such as delusional disorder or schizophrenia; a bipolar disorder, for example bipolar I disorder, bipolar II disorder, and cyclothymic disorder.

This invention further provides a method of treating a neurodegenerative disorder or condition in a mammal, including a human, which method comprises administering to said mammal an amount of a compound of Formulas I or II effective in treating said disorder or condition.

This invention further provides a method of treating a neurodegenerative disorder or condition in a mammal, including a human, which method comprises administering to said mammal an amount of a compound of Formulas I or II effective in inhibiting PDE10.

As used herein, and unless otherwise indicated, a “neurodegenerative disorder or condition” refers to a disorder or condition that is caused by the dysfunction and/or death of neurons in the central nervous system. The treatment of these disorders and conditions can be facilitated by administration of an agent which prevents the dysfunction or death of neurons at risk in these disorders or conditions and/or enhances the function of damaged or healthy neurons in such a way as to compensate for the loss of function caused by the dysfunction or death of at-risk neurons. The term “neurotrophic agent” as used herein refers to a substance or agent that has some or all of these properties.

Examples of neurodegenerative disorders and conditions that can be treated according to the present invention include, but are not limited to, Parkinson's disease; Huntington's disease; dementia, for example Alzheimer's disease, multi-infarct dementia, AIDS-related dementia, and Fronto temperal Dementia; neurodegeneration associated with cerebral trauma; neurodegeneration associated with stroke, neurodegeneration associated with cerebral infarct; hypoglycemia-induced neurodegeneration; neurodegeneration associated with epileptic seizure; neurodegeneration associated with neurotoxin poisoning; and multi-system atrophy.

In one embodiment of the present invention, the neurodegenerative disorder or condition comprises neurodegeneration of striatal medium spiny neurons in a mammal, including a human.

In a further embodiment of the present invention, the neurodegenerative disorder or condition is Huntington's disease.

This invention also provides a pharmaceutical composition for treating psychotic disorders, delusional disorders and drug induced psychosis; anxiety disorders, movement disorders, mood disorders, neurodegenerative disorders and drug addiction, comprising an amount of a compound of Formulas I or II effective in treating said disorder or condition.

This invention also provides a method of treating a disorder selected from psychotic disorders, delusional disorders and drug induced psychosis; anxiety disorders, movement disorders, mood disorders, and neurodegenerative disorders, which method comprises administering an amount of a compound of Formulas I or II effective in treating said disorder.

This invention also provides a method of treating disorders selected from the group consisting of: dementia, Alzheimer's disease, multi-infarct dementia, alcoholic dementia or other drug-related dementia, dementia associated with intracranial tumors or cerebral trauma, dementia associated with Huntington's disease or Parkinson's disease, or AIDS-related dementia; delirium; amnestic disorder; post-traumatic stress disorder; mental retardation; a learning disorder, for example reading disorder, mathematics disorder, or a disorder of written expression; attention-deficit/hyperactivity disorder; age-related cognitive decline, major depressive episode of the mild, moderate or severe type; a manic or mixed mood episode; a hypomanic mood episode; a depressive episode with atypical features; a depressive episode with melancholic features; a depressive episode with catatonic features; a mood episode with postpartum onset; post-stroke depression; major depressive disorder; dysthymic disorder; minor depressive disorder; premenstrual dysphoric disorder; post-psychotic depressive disorder of schizophrenia; a major depressive disorder superimposed on a psychotic disorder comprising a delusional disorder or schizophrenia; a bipolar disorder comprising bipolar I disorder, bipolar II disorder, cyclothymic disorder, Parkinson's disease; Huntington's disease; dementia, Alzheimer's disease, multi-infarct dementia, AIDS-related dementia, Fronto temperal Dementia; neurodegeneration associated with cerebral trauma; neurodegeneration associated with stroke; neurodegeneration associated with cerebral infarct; hypoglycemia-induced neurodegeneration; neurodegeneration associated with epileptic seizure; neurodegeneration associated with neurotoxin poisoning; multi-system atrophy, paranoid, disorganized, catatonic, undifferentiated or residual type; schizophreniform disorder; schizoaffective disorder of the delusional type or the depressive type; delusional disorder; substance-induced psychotic disorder, psychosis induced by alcohol, amphetamine, cannabis, cocaine, hallucinogens, inhalants, opioids, or phencyclidine; personality disorder of the paranoid type; and personality disorder of the schizoid type.

This invention also provides a method of treating psychotic disorders, delusional disorders and drug induced psychosis; anxiety disorders, movement disorders, mood disorders, neurodegenerative disorders and drug addiction which method comprises administering an amount of a compound of Formulas I or II effective in inhibiting PDE10.

This invention also provides a method of treating cancer, preferably renal cancer, which method comprises administering an amount of a compound of Formulas I or II effective in inhibiting PDE10.

The term “alkyl”, as used herein, unless otherwise indicated, includes saturated monovalent hydrocarbon radicals having straight or branched moieties. Examples of alkyl groups include, but are not limited to, methyl, ethyl, propyl, isopropyl, and t-butyl.

The term “alkenyl”, as used herein, unless otherwise indicated, includes alkyl moieties having at least one carbon-carbon double bond wherein alkyl is as defined above.

Examples of alkenyl include, but are not limited to, ethenyl and propeny.

The term “alkynyl”, as used herein, unless otherwise indicated, includes alkyl moieties having at least one carbon-carbon triple bond wherein alkyl is as defined above. Examples of alkynyl groups include, but are not limited to, ethynyl and 2-propynyl. The term “alkoxy”, as used herein, unless otherwise indicated, as employed herein alone or as part of another group refers to an alkyl, groups linked to an oxygen atom.

The term “alkylthio” as used herein, unless otherwise indicated, employed herein alone or as part of another group includes any of the above alkyl groups linked through a sulfur atom.

The term “halogen” or “halo” as used herein alone or as part of another group refers to chlorine, bromine, fluorine, and iodine.

The term “haloalkyl” as used herein, unless otherwise indicated, refers to at least one halo group, linked to an alkyl group. Examples of haloalkyl groups include trifluoromethyl, difluoromethyl and fluoromethyl groups.

The term “cycloalkyl”, as used herein, unless otherwise indicated, includes non-aromatic saturated cyclic alkyl moieties wherein alkyl is as defined above. Examples of cycloalkyl include, but are not limited to, cyclopropyl, cyclobutyl, cyclopentyl, cyclohexyl, and cycloheptyl.

The term “aryl”, as used herein, unless otherwise indicated, includes an organic radical derived from an aromatic hydrocarbon by removal of one hydrogen, such as phenyl, naphthyl, indenyl, and fluorenyl. “Aryl” encompasses fused ring groups wherein at least one ring is aromatic.

The terms “heterocyclic”, “heterocycloalkyl”, and like terms, as used herein, refer to non-aromatic cyclic groups containing one or more heteroatoms, preferably from one to four heteroatoms, each preferably selected from oxygen, sulfur and nitrogen. The heterocyclic groups of this invention can also include ring systems substituted with one or more oxo moieties. Examples of non-aromatic heterocyclic groups are aziridinyl, azetidinyl, pyrrolidinyl, piperidinyl, azepinyl, piperazinyl, 1,2,3,6-tetrahydropyridinyl, oxiranyl, oxetanyl, tetrahydrofuranyl, tetrahydrothienyl, tetrahydropyranyl, tetrahydrothiopyranyl, morpholino, thiomorpholino, thioxanyl, pyrrolinyl, indolinyl, 2H-pyranyl, 4H-pyranyl, dioxanyl, 1,3-dioxolanyl, pyrazolinyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, quinolizinyl, quinuclidinyl, 1,4-dioxaspiro[4.5]decyl, 1,4-dioxaspiro[4.4]nonyl, 1,4-dioxaspiro[4.3]octyl, and 1,4-dioxaspiro[4.2]heptyl.

The term “heteroaryl”, as used herein, refers to aromatic groups containing one or more heteroatoms (preferably oxygen, sulfur and nitrogen), preferably from one to four heteroatoms. A multicyclic group containing one or more heteroatoms wherein at least one ring of the group is aromatic is a “heteroaryl” group. The heteroaryl groups of this invention can also include ring systems substituted with one or more oxo moieties. Examples of heteroaryl groups are pyridinyl, pyridazinyl, imidazolyl, pyrimidinyl, pyrazolyl, triazolyl, pyrazinyl, quinolyl, isoquinolyl, tetrazolyl, furyl, thienyl, isoxazolyl, thiazolyl, oxazolyl, isothiazolyl, pyrrolyl, indolyl, benzimidazolyl, benzofuranyl, cinnolinyl, indazolyl, indolizinyl, phthalazinyl, triazinyl, isoindolyl, purinyl, oxadiazolyl, thiadiazolyl, furazanyl, benzofurazanyl, benzothiophenyl, benzotriazolyl, benzothiazolyl, benzoxazolyl, quinazolinyl, quinoxalinyl, naphthyridinyl, dihydroquinolyl, tetrahydroquinolyl, dihydroisoquinolyl, tetrahydroisoquinolyl, benzofuryl, furopyridinyl, pyrolopyrimidinyl, and azaindolyl.

Unless otherwise indicated, the term “one or more” substituents, or “at least one” substituent as used herein, refers to from one to the maximum number of substituents possible based on the number of available bonding sites.

The term “phenyl, naphthyl, or a 5- to 8-membered heteroaryl ring may be optionally fused to the ring in which R is attached” means that there would be a tricyclic ring group. For example if R is phenyl fused to the ring in which R is attached, the compound of Formula I could have, for example, the structures shown below:

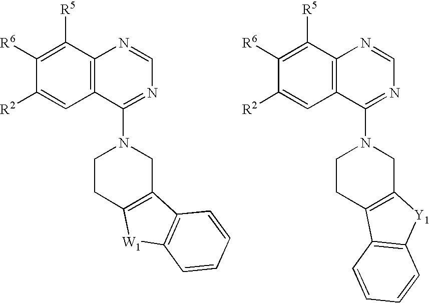

Unless otherwise indicated, all the foregoing groups derived from hydrocarbons may have up to about 1 to about 20 carbon atoms (e.g. C₁-C₂₀ alkyl, C₂-C₂₀ alkenyl, C₃-C₂₀ cycloalkyl, 3-20 membered heterocycloalkyl; C₆-C₂₀ aryl, 5-20 membered heteroaryl, etc.) or 1 to about 15 carbon atoms (e.g., C₁-C₁₅ alkyl, C₂-C₁₅ alkenyl, C₃-C₁₅ cycloalkyl, 3-15 membered heterocycloalkyl, C₆-C₁₅ aryl, 5-15 membered heteroaryl, etc.), or 1 to about 12 carbon atoms, or I to about 8 carbon atoms, or 1 to about 6 carbon atoms.

“Neurotoxin poisoning” refers to poisoning caused by a neurotoxin. A neurotoxin is any chemical or substance that can cause neural death and thus neurological damage. An example of a neurotoxin is alcohol, which, when abused by a pregnant female, can result in alcohol poisoning and neurological damage known as Fetal Alcohol Syndrome in a newborn. Other examples of neurotoxins include, but are not limited to, kainic acid, domoic acid, and acromelic acid; certain pesticides, such as DDT; certain insecticides, such as organophosphates; volatile organic solvents such as hexacarbons (e.g. toluene); heavy metals (e.g. lead, mercury, arsenic, and phosphorous); aluminum; certain chemicals used as weapons, such as Agent Orange and Nerve Gas; and neurotoxic antineoplastic agents.

As used herein, the term “selective PDE10 inhibitor” refers to a substance, for example an organic molecule, that effectively inhibits an enzyme from the PDE10 family to a greater extent than enzymes from the PDE 1-9 families or PDE11 family. In one embodiment, a selective PDE10 inhibitor is a substance, for example an organic molecule, having a Kᵢ for inhibition of PDE10 that is less than or about one-tenth the Kᵢ that the substance has for inhibition of any other PDE enzyme. In other words, the substance inhibits PDE10 activity to the same degree at a concentration of about one-tenth or less than the concentration required for any other PDE enzyme.

In general, a substance is considered to effectively inhibit PDE10 activity if it has a Kᵢ of less than or about 10 μM, preferably less than or about 0.1 μM.

A “selective PDE10 inhibitor” can be identified, for example, by comparing the ability of a substance to inhibit PDE10 activity to its ability to inhibit PDE enzymes from the other PDE families. For example, a substance may be assayed for its ability to inhibit PDE10 activity, as well as PDE1, PDE2, PDE3A, PDE4A, PDE4B, PDE4C, PDE4D, PDE5, PDE6, PDE7, PDE8, PDE9, and PDE11.

The term “treating”, as in “a method of treating a disorder”, refers to reversing, alleviating, or inhibiting the progress of the disorder to which such term applies, or one or more symptoms of the disorder. As used herein, the term also encompasses, depending on the condition of the patient, preventing the disorder, including preventing onset of the disorder or of any symptoms associated therewith, as well as reducing the severity of the disorder or any of its symptoms prior to onset. “Treating” as used herein refers also to preventing a recurrence of a disorder.

For example, “treating schizophrenia, or schizophreniform or schizoaffective disorder” as used herein also encompasses treating one or more symptoms (positive, negative, and other associated features) of said disorders, for example treating, delusions and/or hallucination associated therewith. Other examples of symptoms of schizophrenia and schizophreniform and schizoaffecctive disorders include disorganized speech, affective flattening, alogia, anhedonia, inappropriate affect, dysphoric mood (in the form of, for example, depression, anxiety or anger), and some indications of cognitive dysfunction.

The term “mammal”, as used herein, refers to any member of the class “Mammalia”, including, but not limited to, humans, dogs, and cats.

The compound of the invention may be administered either alone or in combination with pharmaceutically acceptable carriers, in either single or multiple doses. Suitable pharmaceutical carriers include inert solid diluents or fillers, sterile aqueous solutions and various organic solvents. The pharmaceutical compositions formed thereby can then be readily administered in a variety of dosage forms such as tablets, powders, lozenges, liquid preparations, syrups, injectable solutions and the like. These pharmaceutical compositions can optionally contain additional ingredients such as flavorings, binders, excipients and the like. Thus, the compound of the invention may be formulated for oral, buccal, intranasal, parenteral (e.g. intravenous, intramuscular or subcutaneous), transdermal (e.g. patch) or rectal administration, or in a form suitable for administration by inhalation or insufflation.

For oral administration, the pharmaceutical compositions may take the form of, for example, tablets or capsules prepared by conventional means with pharmaceutically acceptable excipients such as binding agents (e.g. pregelatinized maize starch, polyvinylpyrrolidone or hydroxypropyl methylcellulose); fillers (e.g. lactose, microcrystalline cellulose or calcium phosphate); lubricants (e.g. magnesium stearate, talc or silica); disintegrants (e.g. potato starch or sodium starch glycolate); or wetting agents (e.g. sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g. sorbitol syrup, methyl cellulose or hydrogenated edible fats); emulsifying agents (e.g. lecithin or acacia); non-aqueous vehicles (e.g. almond oil, oily esters or ethyl alcohol); and preservatives (e.g. methyl or propyl p-hydroxybenzoates or sorbic acid).

For buccal administration, the composition may take the form of tablets or lozenges formulated in conventional manner.

The compounds of the invention may be formulated for parenteral administration by injection, including using conventional catheterization techniques or infusion. Formulations for injection may be presented in unit dosage form, e.g. in ampules or in multi-dose containers, with an added preservative. They may take such forms as suspensions, solutions or emulsions in oily or aqueous vehicles, and may contain formulating agents such as suspending, stabilizing and/or dispersing agents. Alternatively, the active ingredient may be in powder form for reconstitution with a suitable vehicle, e.g. sterile pyrogen-free water, before use.

When a product solution is required, it can be made by dissolving the isolated inclusion complex in water (or other aqueous medium) in an amount sufficient to generate a solution of the required strength for oral or parenteral administration to patients. The compounds may be formulated for fast dispersing dosage forms (fddf), which are designed to release the active ingredient in the oral cavity. These have often been formulated using rapidly soluble gelatin-based matrices. These dosage forms are well known and can be used to deliver a wide range of drugs. Most fast dispersing dosage forms utilize gelatin as a carrier or structure-forming agent. Typically, gelatin is used to give sufficient strength to the dosage form to prevent breakage during removal from packaging, but once placed in the mouth, the gelatin allows immediate dissolution of the dosage form. Alternatively, various starches are used to the same effect.

The compounds of the invention may also be formulated in rectal compositions such as suppositories or retention enemas, e.g. containing conventional suppository bases such as cocoa butter or other glycerides.

For intranasal administration or administration by inhalation, the compound of the invention is conveniently delivered in the form of a solution or suspension from a pump spray container that is squeezed or pumped by the patient or as an aerosol spray presentation from a pressurized container or a nebulizer, with the use of a suitable propellant, e.g. dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the active compound. Capsules and cartridges (made e.g. from gelatin) for use in an inhaler or insufflator may be formulated containing a powder mix of a compound of the invention and a suitable powder base such as lactose or starch.

Aerosol formulations for treatment of the conditions referred to above (e.g. migraine) in the average adult human are preferably arranged so that each metered dose or “puff” of aerosol contains about 20 mg to about 1000 mg of the compound of the invention. The overall daily dose with an aerosol will be within the range of about 100 mg to about 10 mg. Administration may be several times daily, e.g. 2, 3, 4 or 8 times, giving for example, 1, 2 or 3 doses each time.

A proposed daily dose of the compound of the invention for oral, parenteral, rectal or buccal administration to the average adult human for the treatment of the conditions referred to above is from about 0.01 mg to about 2000 mg, preferably from about 0.1 mg to about 200 mg of the active ingredient of Formulas I or II per unit dose which could be administered, for example, 1 to 4 times per day.

Assay methods are available to screen a substance for inhibition of cyclic nucleotide hydrolysis by the PDE10 and the PDEs from other gene families. The cyclic nucleotide substrate concentration used in the assay is ⅓ of the K, concentration, allowing for comparisons of IC₅₀ values across the different enzymes. PDE activity is measured using a Scintillation Proximity Assay (SPA)-based method as previously described (Fawcett et al., 2000). The effect of PDE inhibitors is determined by assaying a fixed amount of enzyme (PDEs 1-11) in the presence of varying substance concentrations and low substrate, such that the IC₅₀ approximates the Kᵢ (cGMP or cAMP in a 3:1 ratio unlabelled to [³H]-labeled at a concentration of ⅓ Km).). The final assay volume is made up to 100 μl with assay buffer [50 mM Tris-HCl pH 7.5, 8.3 mM MgCl₂, 1 mg/ml bovine serum albumin]. Reactions are initiated with enzyme, incubated for 30-60 min at 30° C. to give <30% substrate turnover and terminated with 50 μl yttrium silicate SPA beads (Amersham) (containing 3 mM of the respective unlabelled cyclic nucleotide for PDEs 9 and 11). Plates are re-sealed and shaken for 20 min, after which the beads were allowed to settle for 30 minutes in the dark and then counted on a TopCount plate reader (Packard, Meriden, Conn.). Radioactivity units can be converted to percent activity of an uninhibited control (100%), plotted against inhibitor concentration and inhibitor IC 50 values can be obtained using the “Fit Curve’ Microsoft Excel extension.

Using such assay, compounds of the present invention were determined to have an IC₅₀ for inhibiting PDE10 activity of less than about 10 micromolar.

This invention also pertains to the preparation of compounds of Formulas I or II.

The schemes below depict various methods of preparing the compounds of the present invention. It should be noted that various substitutents illustrated in the schemes are for illustrated purposes only and should not be confused with and may be independent of those recited above and in the claims.

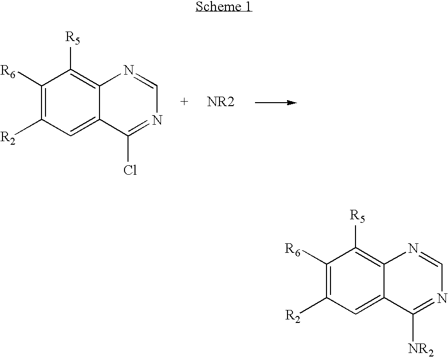

Scheme 1 depicts a coupling reaction between a substituted quinazoline [PC Int. Appl. 2003008388, 30 Jan. 2003] and a secondary amine to form a compound of general formula I and II. This reaction is typically carried out in an inert solvent such as, for example, toluene, optionally in the presence of a carbonate base, at a temperature range of from about 0° C. to about 200° C. Other suitable solvents include benzene, chloroform, dioxane, THF, DMF, ethyl acetate, 2-propanol and xylene. Alternatively, solvent mixtures such as toluene/isopropanol or THF/water can be used. Preferably the reactants are heated under reflux in a solvent mixture of THF and water for a period of from about 2 hours to about 24 hours.

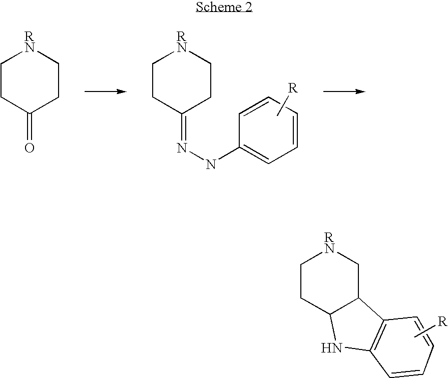

Scheme 2 depicts a method for the generation of 2,3,4,5-tetrahydro-1H-pyrido[4,3-b]indole according to the well known Fischer indole synthesis. In the first step of the method, an N-protected piperidin-4-one is converted into an aryl hydrazone by treatment with an aryl hydrazine. Various N-protecting groups can be employed. Some examples include urethane protecting groups such as the Boc, Cbz, or Fmoc groups. In the second step of the Fisher synthesis, the aryl hydrazone is heated in the presence of Lewis or protic acid catalyst to generate the indole product. In some cases the second step occurs spontaneously to give the indole product directly. Various substituted indoles can be prepared by this method, with the substitution pattern of the product being dictated by the substitution pattern of the starting aryl hydrazine.

The following Examples illustrate the present invention. It is to be understood, however, that the invention, as fully described herein and as recited in the claims, is not intended to be limited by the details of the following Examples.

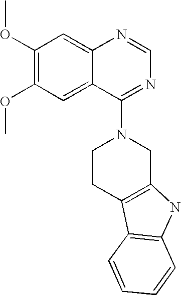

### EXAMPLE 1

2-(6,7-Dimethoxy-quinazolin-4-yl)-2,3,4,9-tetrahydro-1H-b-carboline. To 4-chloro-6,7-dimethoxy quinazoline (239 mg, 1.00 mmol) in toluene (10 mL) and 2-propanol (4 mL) was added 2,3,4,9-tetrahydro-1H-pyrido[3,4-b]indole (206 mg, 1.2 mmol) and K₂CO₃ (280 mg, 2.3 mmol). The mixture was heated to 100° C. until complete by MS analysis. The mixture was diluted with water and the organic portion was dried and concentrated. The residue was chromatographed through silica gel eluting with 10:1 EtOAc/EtOH. The product was converted into the HCl salt via treatment with a solution of HCl in methanol. Crystallization from ethyl acetate yielded 97 mg of a white solid.

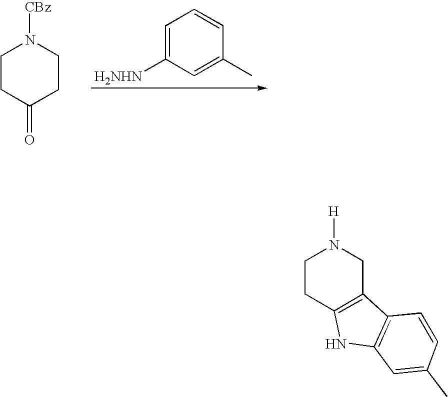

### Preparation 1

7-methyl-2,3,4,5-tetrahydro-1H-pyrido[4,3-b]indole. A mixture of benzyl 4-oxopiperidine-1-carboxylate (116 mg, 0.5 mmol) and 1-m-tolylhydrazine (92 mg (0.6 mmol) in pyridine (2 mL) was heated to reflux for 24 h. The mixture was partitioned between water and CH₂Cl₂, and the organic portion was dried through a cotton plug and purified via passage through silica gel. The resultant oil was then dissolved in ethanol and hydrogenated over 10% palladium on carbon to cleave the Cbz group. The catalyst was carefully filtered away and the product was converted into the HCl salt via treatment with a solution of HCl in ether. Recrystallization from methanol provided the title compound.

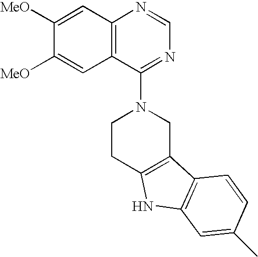

### EXAMPLE 2

2-(6,7-dimethoxyquinazolin-4-yl)-7-methyl-2,3,4,5-tetrahydro-1H-pyrido[4,3-b]indole. Prepared similarly to Example 1.

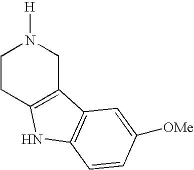

### Preparation 2

8-methoxy-2,3,4,5-tetrahydro-1H-pyrido[4,3-b]indole. Prepared similarly to preparation 1.

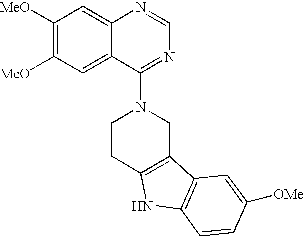

### EXAMPLE 3

2-(6,7-Dimethoxy-quinazolin-4-yl)-8-methoxy-2,3,4,5-tetrahydro-1H-pyrido[4,3-b]indole. Prepared similarly to Example 1.

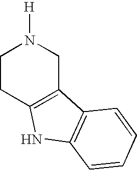

### Preparation 3

2,3,4,5-tetrahydro-1H-pyrido[4,3-b]indole. Prepared similarly to preparation 1.

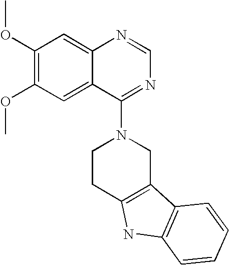

### EXAMPLE 4

2-(6,7-Dimethoxy-quinazolin-4-yl)-2,3,4,5-tetrahydro-1H-pyrido[4,3-b]indole. Prepared similarly to Example 1.

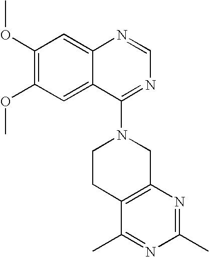

### EXAMPLE 5

7-(6,7-Dimethoxy-quinazolin-4-yl)-2,4-dimethyl-5,6,7,8-tetrahydro-pyrido[3,4-d]pyrimidine. Prepared similarly to Example 1.

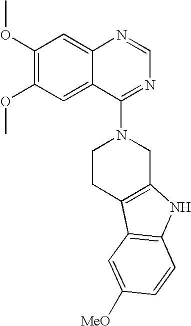

### EXAMPLE 6

2-(6,7-Dimethoxy-quinazolin-4-yl)-6-methoxy-2,3,4,9-tetrahydro-1H-b-carboline repared similarly to Example 1, utilizing commercially available 6-methoxy-2,3,4,9-tetrahydro-1H-pyrido[3,4-b]indole as the amine component.

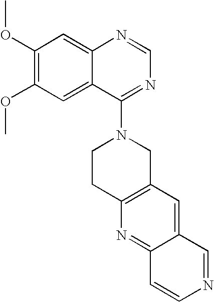

### EXAMPLE 7

2-(6,7-Dimethoxy-quinazolin-4-yl)-1,2,3,4-tetrahydro-2,7,10-triaza-anthracene. Prepared similarly to Example 1 utilizing commercially available 1,2,3,4-tetrahydropyrido[4,3-b][1,6]napthyridine as the amine component.

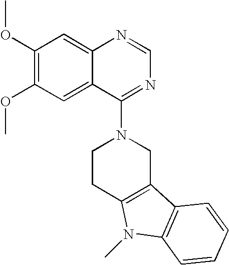

### EXAMPLE 8

2-(6,7-Dimethoxy-quinazolin-4-yl)-5-methyl-2,3,4,5-tetrahydro-1H-pyrido[4.3-b]indole. Prepared similarly to Example 1, utilizing 5-methyl-2,3,4,5-tetrahydro-1H-pyrido[4,3-b]indole (Bioorganic and Medicinal Chemistry, 2003, 11(5) 717-722) as the amine component.

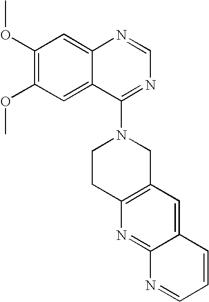

### EXAMPLE 9

6-(6,7-Dimethoxy-quinazolin-4-yl)-5,6,7,8-tetrahydro-1,6,9-triaza-anthracene. Prepared similarly to Example 1, utilizing commercially available 1,2,3,4-tetrahydropyrido[4,3-b][1,8]napthyridine as the amine component.

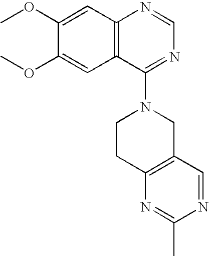

### EXAMPLE 10

6-(6,7-Dimethoxy-quinazolin-4-yl)-2-methyl-5,6,7,8-tetrahydro-pyrido[4,3-d]pyrimidine. Prepared similarly to Example 1, utilizing 2-methyl-5,6,7,8-tetrahydropyrido[4,3-d]pyrimidine (Heterocycles 2002, 56 [1-2] 257-264) as the amine component.

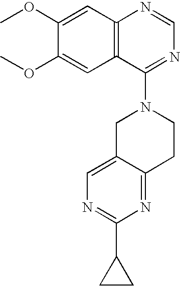

### EXAMPLE 11

3-Cyclopropyl-4-(6,7-dimethoxy-quinazolin-4-yl)-1,3,4,5-tetrahydro-pyrrolo[4,3,2-de]isoquinoline. Prepared similarly to the previous Example.

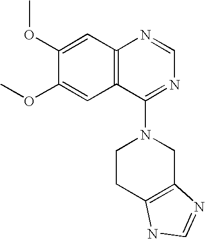

### EXAMPLE 12

6,7-Dimethoxy-4-(1,4,6,7-tetrahydro-imidazo[4,5-c]pyridin-5-yl)-quinazoline. Prepared similarly to Example 1, utilizing 4,5,6,7-Tetrahydro-1H-imidazo[4,5-c]pyridine (ChemBiochem 2004 5 (4) 508-518) as the amine component.

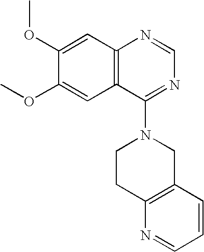

### EXAMPLE 13

4-(7,8-Dihydro-5H-[1,6]naphthyridin-6-yl)-6,7-dimethoxy-quinazoline. Prepared similarly to Example 1, utilizing 5,6,7,8-tetrahydro-1,6-naphthyridine (Chemical and Pharmaceutical Bulletin 1984, 32 [7], 2522-2529) as the amine component.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

1. A compound having the formula I or II: 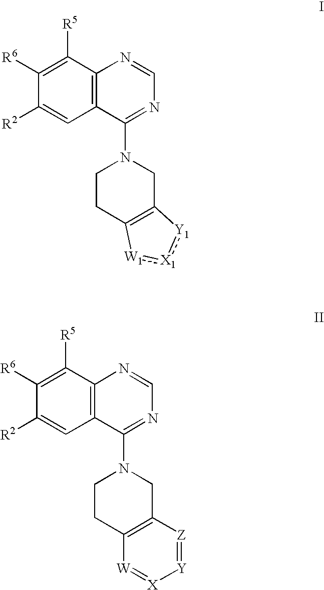
or a pharmaceutically acceptable salt,
wherein the rings containing WI, X₁ and Y₁, and W, X, Y and Z, or tautomers thereof, are aromatic or heteroaromatic;
wherein X₁ is N or CR;
W₁ and Y₁ are each independently N, NR¹ or CR;
wherein the solid and dashed lines between W₁, X₁ and Y₁ represent single or double bonds, provided that one is a single bond and the other is a double bond;
wherein W, X, Y, Z are each independently N or CR;
wherein R², R⁵ and R⁶ are each independently hydrogen, halogen, —CN, —COOH, —COOR³, —CONR³R⁴, —COR³, —NR³R⁴, —OH, —NO₂, —(C₆-C₁₄)aryl, 5 to 12 membered heteroaryl, (C₁-C₈)alkyl, (C₁-C₈)alkoxy (C₂-C₈)alkenyl, (C₂-C₈)alkenyloxy (C₂-C₈)alkynyl or (C₃-C₈) cycloalkyl; wherein said alkyl, alkoxy, alkenyl, alkenyloxy, alkynyl and cycloalkyl are optionally independently substituted with from 1 to 5 halogens; and when R², R⁵ and R⁶ are independently alkoxy, alkenyloxy or alkyl, R² and R⁶ or R⁵ and R⁶ may optionally be connected to form a 5 to 8 membered ring;
wherein each R is independently H, —COOR³, —CONR³R⁴, —COR⁴, —NR³R⁴, —NHCOR³, —OH, —HNCOOR³, —CN, —HNCONHR⁴, (C₁-C₈)alkyl, (C₃-C₈)cycloalkyl, (C₁-C₈)alkoxy, phenyl, naphthyl, or a 5- to 8-membered heteroaryl ring, wherein said phenyl, naphthyl, or a 5- to 8-membered heteroaryl ring may be optional fused to the ring in which R is attached and wherein said alkyl, cycloalkyl, alkoxy, phenyl, naphthyl, heteroaryl or fused ring may optionally be substituted with from one to three substituents independently selected from (C₁-C₈)alkyl, (C₁-C₈)alkoxy, halogen, halo(C₁-C₅)alkyl, halo(C₁-C₈)alkoxy, (C₁-C₈)hydroxyalkyl, (C₁-C₈)alkoxy-(C₁-C₈)alkyl, (C₃-C₈)hydroxycycloalkyl, (C₃-C₈)cycloalkoxy, (C₁-C₈)alkoxy-(C₃-C₈)cycloalkyl, heterocycloalkyl, hydroxyheterocycloalkyl, and (C₁-C₈)alkoxy-heterocycloalkyl, wherein each cycloalkyl or heterocycloalkyl moiety may be independently substituted with from one to three (C₁-C₆)alkyl or benzyl groups;
wherein R¹ is H, —COOR³, —CONR³R⁴, —COR⁴, (C₁-C₈)alkyl, (C₃-C₈)cycloalkyl, phenyl, naphthyl, or a 5- to 8-membered heteroaryl ring, wherein said alkyl, cycloalkyl, phenyl, naphthyl, heteroaryl may optionally be substituted with from one to three substituents independently selected from (C₁-C₈)alkyl, (C₁-C₈)alkoxy, halogen, halo(C₁-C₈)alkyl, halo(C₁-C₈)alkoxy, (C₁-C₈)hydroxyalkyl, (C₁-C₈)alkoxy-(C₁-C₈)alkyl, (C₃-C₈)hydroxycycloalkyl, (C₃-C₈)cycloalkoxy, (C₁-C₈)alkoxy-(C₃-C₈)cycloalkyl, heterocycloalkyl, hydroxyheterocycloalkyl, and (C₁-C₈)alkoxy-heterocycloalkyl, wherein each cycloalkyl or heterocycloalkyl moiety may be independently substituted with from one to three (C₁-C₆)alkyl or benzyl groups;
wherein R³ and R⁴ are each independently H, (C₁-C₈)alkyl, alkenyl, aryl or benzyl; or R³ and R⁴ together with the nitrogen to which they are attached form a 5- to 8-membered heteroalkyl ring.

2. The compound of claim 1, wherein R is phenyl or pyridine fused to the ring in which R is attached; W₁ or Y₁ is N or NR¹ in Formula I; or W or Y is N in Formula II.

3. The compound of claim 2 wherein R is further substituted by (C₁-C₄)alkoxy, (C₁-C₄)alkyl, (C₁-C₅)trifluoroalkyl or (C₁-C₅)trifluoroalkoxy.

4. The compound of claim 1 wherein R is H, (C₁-C₆)alkoxy, (C₁-C₆)alkyl, (C₁-C₅)trifluoroalkyl, (C₁-C₅)trifluoroalkoxy or phenyl; R¹ is H, (C₁-C₆)alkyl, (C₁-C₅)trifluoroalkyl, or phenyl and at least one of W, X, Y, Z are nitrogen in Formula II.

5. The compound of claim 1 wherein R⁶ and R² are each independently (C₁-C₆)alkoxy.

6. The compound of claim 1 wherein R⁶ and R² are each independently ethoxy or methoxy.

7. The compound of claim 2 wherein the compound is selected from formula I and R⁶ and R² are each independently (C₁-C₆)alkoxy.

8. The compound of claim 2 wherein the compound is selected from formula II and R⁶ and R² are each independently (C₁-C₆)alkoxy.

9. The compound of claim 4 wherein R⁶ and R² are each independently (C₁-C₆)alkoxy.

10. A compound according to claim 1 selected from the group consisting of:
2-(6,7-Dim ethoxy-quinazolin-4-yl)-8-methoxy-2,3,4,5-tetrahydro-1H-pyrido[4,3-b]indole;
2-(6,7-Dimethoxy-quinazolin-4-yl)-2,3,4,5-tetrahydro-1H-pyrido[4,3-b]indole;
7-(6,7-Dimethoxy-quinazolin-4-yl)-2,4-dimethyl-5,6,7,8-tetrahydro-pyrido[3,4-d]pyrimidine;
2-(6,7-Dimethoxy-quinazolin-4-yl)-2,3,4,9-tetrahydro-1H-b-carboline;
2-(6,7-Dimethoxy-quinazolin-4-yl)-6-methoxy-2,3,4,9-tetrahydro-1H-b-carboline;
2-(6,7-Dimethoxy-quinazolin-4-yl)-1,2,3,4-tetrahydro-2,7,10-triaza-anthracene;
2-(6,7-Dimethoxy-quinazolin-4-yl)-5-methyl-2,3,4,5-tetrahydro-1H-pyrido[4,3-b]indole;
6-(6,7-Dimethoxy-quinazolin-4-yl)-5,6,7,8-tetrahydro-1,6,9-triaza-anthracene;
6-(6,7-Dimethoxy-quinazolin-4-yl)-2-methyl-5,6,7,8-tetrahydro-pyrido[4,3-d]pyrimidine;
6,7-Dimethoxy-4-(1,4,6,7-tetrahydro-imidazo[4,5-c]pyridin-5-yl)-quinazoline;
4-(7,8-Dihydro-5H-[1,6]naphthyridin-6-yl)-6,7-dimethoxy-quinazoline;
3-Cyclopropyl-4-(6,7-dimethoxy-quinazolin-4-yl)-1,3,4,5-tetrahydro-pyrrolo[4,3,2-de]isoquinoline;
2-(6,7-dimethoxyquinazolin-4-yl)-7-methyl-2,3,4,5-tetrahydro-1H-pyrido[4,3-b]indole;
and pharmaceutical acceptable salts thereof.

11. A pharmaceutical composition for treating psychotic disorders, delusional disorders and drug induced psychosis; anxiety disorders, movement disorders, obesity, cancer, mood disorders, neurodegenerative disorders and drug addiction, comprising an amount of a compound of Formulas I or II according to claim 1 effective in treating said disorder or condition.

12. A method of treating a disorder selected from psychotic disorders, delusional disorders and drug induced psychosis; anxiety disorders, movement disorders, obesity, cancer, mood disorders, and neurodegenerative disorders, which method comprises administering an amount of a compound of Formulas I or II according to claim 1 effective in treating said disorder.

13. The method of claim 12, wherein said disorder are selected from the group consisting of: dementia, Alzheimer's disease, multi-infarct dementia, alcoholic dementia or other drug-related dementia, dementia associated with intracranial tumors or cerebral trauma, dementia associated with Huntington's disease or Parkinson's disease, or AIDS-related dementia; delirium; amnestic disorder; post-traumatic stress disorder; mental retardation; a learning-disorder, for example reading disorder, mathematics disorder, or a disorder of written expression; attention-deficit/hyperactivity disorder; age-related cognitive decline, major depressive episode of the mild, moderate or severe type; a manic or mixed mood episode; a hypomanic mood episode; a depressive episode with atypical features; a depressive episode with melancholic features; a depressive episode with catatonic features; a mood episode with postpartum onset; post-stroke depression; major depressive disorder; dysthymic disorder; minor depressive disorder; premenstrual dysphoric disorder; post-psychotic depressive disorder of schizophrenia; a major depressive disorder superimposed on a psychotic disorder comprising a delusional disorder or schizophrenia; a bipolar disorder comprising bipolar I disorder, bipolar II disorder, cyclothymic disorder, Parkinson's disease; Huntington's disease; dementia, Alzheimer's disease, multi-infarct dementia, AIDS-related dementia, Fronto temperal Dementia; neurodegeneration associated with cerebral trauma; neurodegeneration associated with stroke; neurodegeneration associated with cerebral infarct; hypoglycemia-induced neurodegeneration; neurodegeneration associated with epileptic seizure; neurodegeneration associated with neurotoxin poisoning; multi-system atrophy, paranoid, disorganized, catatonic, undifferentiated or residual type; schizophreniform disorder; schizoaffective disorder of the delusional type or the depressive type; delusional disorder; substance-induced psychotic disorder, psychosis induced by alcohol, amphetamine, cannabis, cocaine, hallucinogens, obesity, renal cancer, inhalants, opioids, or phencyclidine; personality disorder of the paranoid type; and personality disorder of the schizoid type.
